(19) United States
(12) Patent Application Publication
(10) Pub. No.: US 2024/0281713 A1
(43) Pub. Date: Aug. 22, 2024

(54) INTELLIGENT DETECTION OF FIBER COMPOSITION OF TEXTILES THROUGH AUTOMATED ANALYSIS BY MACHINE LEARNING MODELS

(71) Applicant: Refiberd, Inc., Cupertino, CA (US)

(72) Inventors: Tushita Gupta, Dublin, CA (US); Sarika Bajaj, Cupertino, CA (US); Mingyue Wang, Sunnyvale, CA (US)

(21) Appl. No.: 18/639,019

(22) Filed: Apr. 18, 2024

Related U.S. Application Data
(63) Continuation of application No. PCT/US22/78233, filed on Oct. 17, 2022.
(60) Provisional application No. 63/257,055, filed on Oct. 18, 2021.

(51) Int. Cl. G06N 20/00

(52) U.S. Cl. CPC G06N 20/00

(57) ABSTRACT

Introduced here is a process for training a machine learning model to predict a characteristic of a textile through analysis of spectral information. The spectral information may include spectra generated by a spectroscopy instrument, for example. The machine learning model can be used for a variety of applications, including automated sorting of textiles for recycling and automated analyzing of textiles for quality control.

### CROSS-REFERENCE TO RELATED APPLICATIONS

This application is a continuation of International Patent Application No. PCT/US2022/078233, filed Oct. 17, 2022, which claims the benefit of priority to U.S. Provisional Application No. 63/257,055, filed Oct. 18, 2021, each of which is incorporated herein by reference in its entirety.

### TECHNICAL FIELD

The present invention relates to approaches to training a machine learning model to predict characteristics of textiles and implementing the same.

### BACKGROUND

The term “textile” is commonly used to refer to a flexible material that is made by creating an interlocking network of yarns (also called “threads”). Yarns are generally produced by spinning raw fibers-made from either natural or synthetic materials-into long, twisted lengths. Textiles are then formed by weaving, knitting, crocheting, knotting, tatting, felting, bonding, or braiding yarns together. Thus, the term “textile” may be used to refer to any flexible material made of interlacing fibers. Note that the terms “fabric,” “cloth,” and “material” are often used in textile-related trades as synonyms for the term “textile.” While there are subtle differences in the meanings of these terms, those skilled in the art will recognize that these terms tend to be used interchangeably.

Textiles have a broad assortment of uses, from clothing, carpeting, and coverings (e.g., for tablets, beds, and the like) to flags, tents, and towels. Regardless of application, textiles tend to have a relatively brief lifespan when used in a repeated or consistent manner. As an example, articles of clothing made from textiles will become worn out or damaged over time. While some unwanted articles are directed to second-hand retailers (also called “resale retailers” or “thrift retailers”) to be resold, the vast majority of unwanted articles are eventually found in landfills.

Recycling represents an attractive solution to this problem. Through recycling, the fibers of a textile can be recovered and then reprocessed into another useful textile product (or simply “product”). The recycling process generally involves gathering products from different sources and then manually sorting and processing those products based on factors such as condition, composition, and the like. The end result of the recycling process can vary depending on the desired output. For example, those products may be deconstructed to produce chemicals, or those products may be dismantled into the fibers from which new products can be created. While several entities have attempted to develop approaches for dealing with textiles through recycling, adoption has been limited because the practicality of these approaches is limited. In particular, these approaches tend not to be time—or resource-efficient as the acquiring, sorting, and processing of textiles is normally very labor intensive.

### BRIEF DESCRIPTION OF THE DRAWINGS

FIG. 1 includes a high-level overview of a process for recovering the fibers of a textile and then reprocessing the recovered fibers into useful products.

FIG. 2 illustrates a network environment that includes an analysis platform that is executed by a computing device.

FIG. 3 illustrates an example of a computing device that is able to implement an analysis platform designed to train a model to characterize a textile based on analysis of corresponding spectral information and apply the model to accomplish the same.

FIG. 4 depicts an example of a communication environment that includes an analysis platform that is configured to obtain data from one or more sources.

FIG. 5 includes a flow diagram of a process for training and then implementing a model to determine fiber composition.

FIG. 6 includes a flow diagram of a process for training a model to predict a characteristic of a textile via spectral analysis.

FIG. 7 includes a flow diagram of a process for implementing a trained model to predict a characteristic of a textile via spectral analysis.

FIG. 8 includes a block diagram illustrating an example of a processing system in which at least some operations described herein can be implemented.

Embodiments are illustrated by way of example and not limitation in the drawings. While the drawings depict various embodiments for the purpose of illustration, those skilled in the art will recognize that alternative embodiments may be employed without departing from the principles of the technology. Accordingly, while specific embodiments are shown in the drawings, the technology is amenable to various modifications.

### DETAILED DESCRIPTION OF THE INVENTION

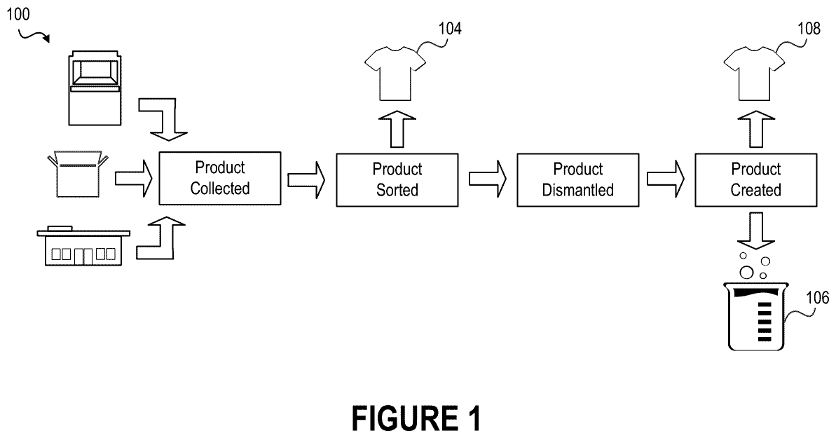

Through recycling, the fibers of a textile can be recovered and then reprocessed into useful products. As shown in FIG. 1, products are generally gathered from different sources 100—such as collection boxes and resale organizations—and then manually sorted and processed depending on condition, composition, and resale value. Products 104 with high resale value may be set aside for resale, while products with low resale value may be deconstructed or dismantled. For example, products that are not suitable for resale may be deconstructed to produce chemicals 106 or dismantled into fibers from which new products 108 can be created.

Recycling has become a focus of sustainability efforts, as overconsumption and waste generation continue to plague the textile industry. As an example, globalization has led to a “fast fashion” trend where clothes are considered disposable by many consumes due to increasingly lower prices. This has allowed—and perhaps incentivized—manufacturers to produce vast amounts of products that deplete natural resources.

Several entities have attempted to develop approaches for dealing with textiles through recycling. Now, even manufacturers and retailers have begun embracing recycling efforts, for example, by advertising products that have been “upcycled.” The term “upcycled product” may be used to refer to products that are sorted out and then resold and products that are created from reclaimed fibers.

These conventional approaches to recycling tend to be costly in terms of time and resources, however. Specifically, aspects of the sorting process tend to be very labor intensive. Historically, products have not only been manually sorted, for example, to distinguish articles of clothing from one another, but also manually analyzed for quality control purposes. As an example, an individual may be responsible for sorting pairs of jeans from among the vast amounts of clothes collected from various sources 100 and then determining whether each pair of jeans is suitable for resale or dismantling.

Introduced here is a process for intelligently detecting the fiber composition of textiles using a machine learning model (or simply “model”). The model can be used for a variety of applications, including automated sorting of textiles for recycling and automated analyzing of textiles for quality control. Accordingly, while the process may be described in the context of recycling, those skilled in the art will recognize that aspects of the process may be applicable to other contexts.

The process involves two stages, namely, (i) training the model and (ii) implementing the model.

In the training stage, a labeled dataset is initially created for textiles covering different fibers, blend ratios, pre-treatments (or simply “treatments”), colors, ages, wear-and-tear levels, thicknesses, layer counts, and the like. The breadth and depth of the labeled dataset may depend on the intended application of the model. As an example, an individual (also referred to as a “trainer” or “administrator”) may obtain a set of textiles where different fibers are represented in various blend ratios (e.g., 100 percent cotton, 50 percent cotton and 50 percent polyester, 30 percent rayon and 70 percent nylon). To ensure that the model is able to appropriately generalize, a wide gamut of fibers and blend ratios are normally covered. Accordingly, the individual may ensure that a range of different blend ratios is covered at sufficiently small intervals to provide insight into the distinctions between those different blend ratios. As an example, the set may include textiles that are 100 percent cotton, 95 percent cotton, 90 percent cotton, etc. Similarly, the set may include textiles that are 80 percent cotton and 20 percent polyester, 80 percent cotton and 20 percent nylon, etc. The set of textiles may include tens, hundreds, or thousands of different textiles that cover different fibers, blend ratios, treatments, colors, ages, wear-and-tear levels, thicknesses, layer counts, and the like.

In some embodiments, a sufficient number of textiles are included in the set so as to allow the characteristics of non-examined blend ratios to be interpolated. Assume, for example, that the set includes one or more textiles having a blend ratio of 10 percent cotton and 90 percent polyester and one or more textiles having a blend ratio of 20 percent cotton and 80 percent polyester. As further discussed below, spectral information can be obtained for these textiles, such that a computer program can programmatically establish the spectral characteristics of two different blend ratios. The computer program may be referred to as a “textile analysis platform” or simply “analysis platform.” Through interpolation, the analysis platform may be able to infer the spectral characteristics of a non-examined blend ratio between the examined blend ratios. Referring again to the aforementioned example, the analysis platform may be able to infer the spectral characteristics of a textile that is 15 percent cotton and 85 percent polyester based on an analysis (e.g., a weighted analysis) of (i) the spectral characteristics of the textiles that are 10 percent cotton and 90 percent polyester and (ii) the spectral characteristics of the textiles that are 20 percent cotton and 80 percent polyester.

Given the broad range of textiles that are available in commerce, interpolation allows the analysis platform to better accommodate textiles whose characteristics are unknown. In that sense, interpolation makes the analysis platform more “elastic.” Simply put, with interpolation—or another form of numerical analysis—the analysis platform can more quickly analyze new combinations of fibers, for example, since a matching example does not need to be added to the labeled dataset used for training.

Each textile included in the set can be measured using a spectrometry instrument to create corresponding spectral information. For example, the individual may use a spectrometer or hyperspectral camera to measure spectral components of each textile. As a specific example, the individual may use a near infrared spectrometer (also called a “NIR spectrometer”) to obtain spectral information for a textile of interest. As another specific example, the individual may use a hyperspectral camera that acts as a line-scan imaging instrument by scanning through the visible, ultraviolet, and near-infrared spectral ranges to obtain spectral information for a textile of interest. In embodiments where the spectrometry instrument relies on visible light, individual bands of color-called a “spectrum”—can be measured for each textile. The spectral information associated with each textile may include the spectrum measured for that textile. Additionally or alternatively, the spectral information associated with each textile may include information learned, derived, or otherwise determined through analysis of the spectrum measured for that textile. Note that while embodiments may be described in the context of spectrometers for the purpose of illustration, those skilled in the art will recognize that the features of those embodiments may be similarly applicable to hyperspectral cameras and other types of spectrometry instruments.

This “raw” spectral information can then be processed in preparation for training. For example, the spectral information could be normalized, filtered, or otherwise manipulated by the analysis platform to ensure that the spectral information is suitable for training. As a specific example, the analysis platform may perform principal component analysis (“PCA”) to reduce the dimensionality of the spectral information. PCA is a popular technique for analyzing datasets that contain a high number of dimensions, increasing interpretability of the multidimensional data contained therein while preserving as much information as possible, and enabling the visualization of the multidimensional data. As another specific example, the analysis platform may apply a digital filter (or simply “filter”), such as a Savitzky-Golay filter, to the spectral information for the purpose of “smoothing” the corresponding data. By “smoothing” the corresponding data, the analysis platform can increase precision of the corresponding data without meaningfully distorting the tendency of the actual values.

The analysis platform can then train a model using the labeled dataset that includes, for each textile included in the set, (i) a corresponding blend ratio and (ii) corresponding spectral information. Specifically, the analysis platform can provide the labeled dataset to a model as training data, so as to produce a trained model that is able to produce, as output, a predicted blend ratio for a textile having an unknown blend ratio based on spectral information measured, generated, or otherwise obtained for the textile. Thus, the trained model may be operable to produce, as output, predicted fiber composition, predicted blend ratio, predicted treatment, predicted color, predicted age, predicted wear-and-tear level, predicted thickness, predicted layer counts, or any combination thereof. The nature and number of the outputs will depend on the contents of the labeled dataset.

In the implementing stage, a textile having an unknown blend ratio may initially be obtained. Assume, for example, that an entity comes into possession of a textile to be recycled. In such a scenario, the entity may cause a spectrum of the textile to be measured through spectral analysis by a spectrometer or another imaging instrument. The analysis platform can then apply the trained model to the spectrum, so as to produce an output that is representative of a prediction. As noted above, the prediction may correspond to fiber composition, blend ratio, treatment, color, age, wear-and-tear level, thickness, layer count, or some combination thereof. The spectrum may be one of multiple spectra that are measured through spectral analysis by the spectrometer as further discussed below. In embodiments where the spectrum is one of multiple spectra measured for the textile, the trained model may produce multiple outputs, each of which is representative of a separate prediction by the trained model based on a corresponding spectrum of the multiple spectra. Assume, for example, that the analysis platform is tasked with determining fiber composition of the textile. In the event that the analysis platform obtains multiple outputs, each of which is representative of a separate prediction regarding fiber composition, the analysis platform may deterministically average the multiple outputs to determine the fiber composition of the textile.

Those skilled in the art will recognize that the outputs produced by the trained model can be used in several different ways. For example, a predicted wear-and-tear level may be used to determine whether a given textile should be set aside for resale or set aside for dismantling. As another example, a predicted blend ratio may be used to determine how to sort a given textile amongst different collections of textiles to be recycled that correspond to different blend ratios. A predicted blend ratio could also be used to determine how to handle a given textile. For example, textiles having some blend ratios may be candidates for dismantling (e.g., into reusable fibers), while textiles having other blend ratios may be candidates for deconstructing (e.g., into chemicals).

### Terminology

Brief definitions of some terms used in the present disclosure are provided below.

The term “textile” may be used to refer to any flexible material that is made by creating an interlocking network of threads comprised of fibers. The term “fibers” may be used to refer to the smallest unit of a textile, as well as the resultant after the textile is deconstructed. Fibers can be spun, linked, or otherwise connected to create “threads.” Meanwhile, threads can be woven, knitted, or otherwise connected to make “textiles.”

The terms “connected,” “coupled,” and any variants thereof are intended to include any connection or coupling between two or more elements, either direct or indirect. The connection or coupling can be physical, logical, or a combination thereof. For example, objects may be electrically or communicatively connected to one another despite not sharing a physical connection.

The term “module” may be used to refer broadly to components implemented via software, firmware, hardware, or any combination thereof. Generally, modules are functional components that generate one or more outputs based on one or more inputs. A computer program—like the analysis platform—may include modules that are responsible for completing different tasks, though those modules may work in concert with one another (e.g., the output produced by one module may be provided to another module as input).

### Overview of Analysis Platform

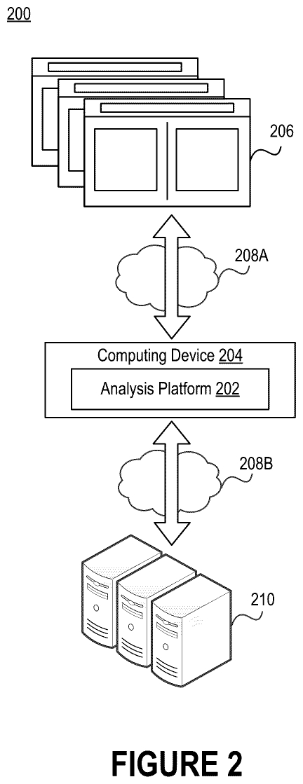

FIG. 2 illustrates a network environment 200 that includes an analysis platform 202 that is executed by a computing device 204. An individual (also called a “user”) may be able to interact with the analysis platform 202 via interfaces 206. For example, a user may be able to access an interface through which she can create a labeled dataset to be used to train a model. As another example, a user may be able to access an interface through which she can review outputs produced by a model upon being applied to spectral information associated with a textile.

As shown in FIG. 2, the analysis platform 202 may reside in a network environment 200. Thus, the computing device 204 on which the analysis platform 202 resides may be connected to one or more networks 208A-B. Depending on its nature, the computing device 204 could be connected to a personal area network (“PAN”), local area network (“LAN”), wide area network (“WAN”), metropolitan area network (“MAN”), or cellular network. For example, if the computing device 204 is a computer server, then the computing device 204 may be accessible to users via respective computing devices that are connected to the Internet via LANs.

The interfaces 206 may be accessible via a web browser, desktop application, mobile application, or another form of computer program. For example, to interact with the analysis platform 202, a user may initiate a web browser and then navigate to a web address associated with the analysis platform 202. As another example, a user may access, via a mobile application or desktop application, interfaces that are generated by the analysis platform 202 through which she can select spectral information for inclusion in the labeled dataset used for training, select spectral information to which a model is to be applied, review outputs produced by the model, and the like. Accordingly, interfaces generated by the analysis platform 202 may be accessible via various computing devices, including mobile phones, tablet computers, desktop computers, and the like.

Generally, the analysis platform 202 is executed by a cloud computing service operated by, for example, Amazon Web Services®, Google Cloud Platform™, or Microsoft Azure®. Thus, the computing device 204 may be representative of a computer server that is part of a server system 210. Often, the server system 210 is comprised of multiple computer servers. These computer servers can include different types of data (e.g., spectral information for different textile samples and characteristics of those different textile samples, such as blend ratio, treatment, color, etc.), algorithms for processing spectral information, models trained to surface insights through analysis of spectral information, and other assets. Those skilled in the art will recognize that this data could also be distributed among the server system 210 and other computing devices. For example, spectral information associated with a textile whose characteristics are unknown may be stored on, and initially processed by, a computing device that is responsible for generating the spectral information. The computing device could be a spectrometer or another imaging instrument, such as a mobile phone, tablet computer, etc. This spectral information could be processed before being transmitted to the server system 210 for further processing. Alternatively, the computing device may receive, from the server system 210, a model that can be applied to the spectral information, so that insights can be surfaced locally.

As mentioned above, aspects of the analysis platform 202 could be hosted locally, for example, in the form of a computer program executing on the computing device 204. Several different versions of computer programs may be available depending on the intended use. Assume, for example, that a user would like to actively guide the process by which a model is trained to predict characteristics of a textile through analysis of spectral information. In such a scenario, the computer program may allow for the selection of textiles or corresponding spectral information to be used for training. Moreover, the computer program may allow the user to manage or review testing of the model on another labeled dataset. This other labeled dataset —commonly called the “testing dataset”—may be representative of a subset of the training dataset, or the testing dataset may be different than the training dataset. Notably, for each textile included in the testing dataset, the characteristics are already known, so the user can manually determine whether the model is predicting appropriate characteristics. Alternatively, the analysis platform 202 may automatically monitor whether the model is predicting appropriate characteristics by comparing its outputs to the labels included in the testing dataset. As another example, if the user is simply interested in reviewing analyses of outputs produced by a model upon being applied to spectral information, the computer program may be “simpler.”

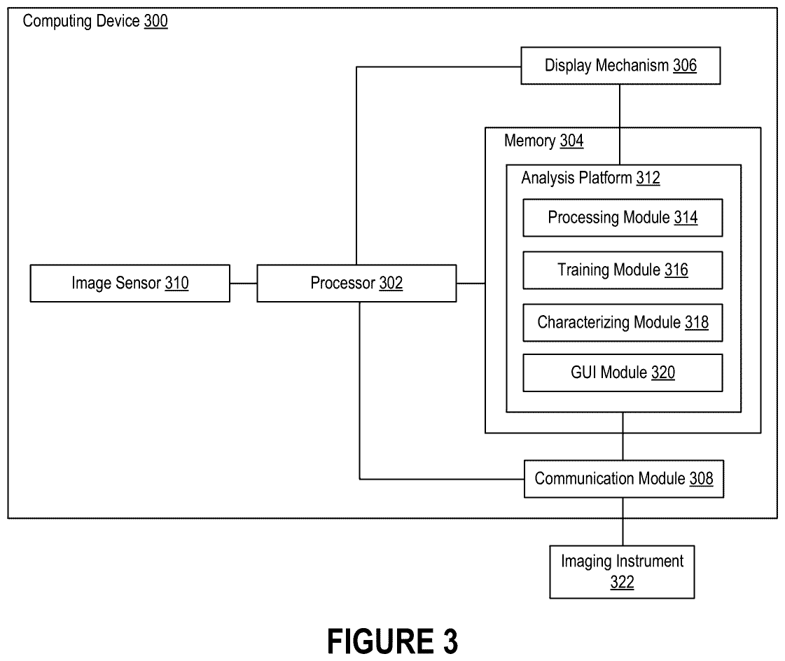

FIG. 3 illustrates an example of a computing device 300 that is able to implement an analysis platform 312 designed to train a model to characterize a textile based on analysis of corresponding spectral information and apply the model to accomplish the same. Note that embodiments of the analysis platform 312 could be designed to train the model, apply the model, or train and apply the model. Accordingly, the nature of the analysis platform, and specifically its modules, may vary depending on the intended responsibilities of the analysis platform 312.

As shown in FIG. 3, the computing device 300 can include a processor 302, memory 304, display mechanism 306, communication module 308, and image sensor 310. Each of these components is discussed in greater detail below. Those skilled in the art will recognize that different combinations of these components may be present depending on the nature of the computing device 300. For example, if the computing device 300 is a computer server that is part of a server system (e.g., server system 210 of FIG. 2), then the computing device 300 may not include the display mechanism 306 or image sensor 310. Instead, spectral information may be obtained from an imaging instrument 322 that is external to the computing device 300. The imaging instrument 322 could be a spectrometer, mobile phone, or tablet computer, for example.

The processor 302 can have generic characteristics similar to general-purpose processors, or the processor 302 may be an application-specific integrated circuit (“ASIC”) that provides control functions to the computing device 300. The processor 302 can be coupled to all components of the computing device 300, either directly or indirectly, for communication purposes.

The memory 304 may be comprised of any suitable type of storage medium, such as static random-access memory (“SRAM”), dynamic random-access memory (“DRAM”), electrically erasable programmable read-only memory (“EEPROM”), flash memory, or registers. In addition to storing instructions that can be executed by the processor 302, the memory 304 can also store data to be used or generated by the processor 302 (e.g., when executing the modules of the analysis platform 310). As an example, the memory 304 may include a data structure that has a plurality of entries, each of which includes spectral information and specifies one or more characteristics of a corresponding textile. This data structure may be used to train the model, as further discussed below. As another example, when the analysis platform 312 applies a model to spectral information associated with a textile having unknown characteristics, any insights surfaced by the model may be populated into a data structure that is representative of a digital profile (or simply “profile”) created for the textile. Note that the memory 304 is merely an abstract representation of a storage environment. The memory 304 could be comprised of actual integrated circuits (also called “chips”).

The display mechanism 306 can be any mechanism that is operable to visually convey information to a user. For example, the display mechanism 306 may be a panel that includes light-emitting diodes (“LEDs”), organic LEDs, liquid crystal elements, or electrophoretic elements. In some embodiments, the display mechanism 306 is touch sensitive. Thus, the user may be able to provide input to the analysis platform 210 312 by interacting with the display mechanism 306. Alternatively, the user may be able to provide input to the analysis platform 312 through some other control mechanism.

The communication module 308 may be responsible for managing communications external to the computing device 300. The communication module 308 may be wireless communication circuitry that is able to establish wireless communication channels with other computing devices. Examples of wireless communication circuitry include 2.4 gigahertz (“GHz”) and 5 GHz chipsets compatible with Institute of Electrical and Electronics Engineers (“IEEE”) 802.11—also referred to as “Wi-Fi chipsets.” Alternatively, the communication module 308 may be representative of a chipset configured for a short-range communication protocol such as Bluetooth® or Near Field Communication. Some computing devices-like mobile phones, tablet computers, and the like—are able to wirelessly communicate via different types of channels. Accordingly, the communication module 308 may be one of multiple communication modules implemented in the computing device 300. Other computing devices-like computer servers—are generally designed to wirelessly communicate via a single type of channel.

In some embodiments, communications transmitted or received by the communication module 308 are formatted in accordance with the MQTT protocol. The processor 302, rather than the communication module 308, may be responsible for formatting messages in accordance with the MQTT protocol. The MQTT format has a 2-byte fixed header that is always present, another header having variable length that is optional, and a payload having variable length that is optional. Accordingly, possible formats include (i) fixed header only, (ii) fixed header plus variable header, and (iii) fixed header plus variable header plus variable payload. The fixed header can include the control field (e.g., 1 byte) and the packet length field (e.g., 1-4 bytes). As mentioned above, the variable header may not always be present in an MQTT message. For example, only some MQTT message types may use the variable header to carry additional control information. Meanwhile, the variable payload includes the data being sent via the communication module 308. Formatting outgoing messages in accordance with the MQTT protocol may be useful for communicating spectral information or other outputs (e.g., analyses of spectral information) with other computing devices, storage mediums, robotic computing systems, and the like.

The nature, number, and type of communication channels established by the computing device 300—and more specifically, the communication module 308—may depend on the sources from which data is acquired by the analysis platform 312. Assume, for example, that the analysis platform 312 resides on a computer server of a server system (e.g., server system 210 of FIG. 2). In such embodiments, the communication module 308 may communicate with databases from which to obtain spectral information, or the communication module 308 may communicate with the imaging instruments (e.g., imaging instrument 322) that are responsible for generating the spectral information.

For convenience, the analysis platform 312 is referred to as a computer program that resides in the memory 304. However, the analysis platform 312 could be comprised of hardware or firmware in addition to, or instead of, software. In accordance with embodiments described herein, the analysis platform 312 may include a processing module 314, training module 316, characterizing module 318, and graphical user interface (“GUI”) module 320. These modules can be an integral part of the analysis platform 312. Alternatively, these modules can be logically separate from the analysis platform 312 but operate “alongside” it. Together, these modules may enable the analysis platform 312 to train models to characterize textiles through analysis of corresponding spectral information. Said another way, the models may be able to surface insights into characteristics of textiles through spectral analysis. As further discussed below, the model may be in the form of a regression model that is based on, for example, a random forest, neural network, dense or deep neural network, linear regression, or support vector machine. Alternatively, the model may be in the form of a classification model, for example, that is trained to determine whether there are—or are not—fibers present in the item undergoing analysis.

The processing module 314 may be responsible for applying operations to data that is acquired by the analysis platform 312. Assume, for example, that the analysis platform 312 receives input indicative of selection of a plurality of imaging instruments from which to obtain spectral information. The processing module 314 may process (e.g., filter, reorder, or otherwise alter) spectral information so that it is usable by the other modules of the analysis platform 312. Approaches to processing spectral information, for example, in preparation for training of a model, are further discussed below.

The training module 316 may be responsible for training a model to establish characteristics of a textile through analysis of its spectral information. Initially, the training module 316 can receive input indicative of a request to train a model to predict a characteristic of a textile via spectral analysis. Thereafter, the training module 316 can obtain a labeled dataset covering different fibers, blend ratios, treatments, colors, ages, wear-and-tear levels, thicknesses, layer counts, or any combination thereof. In some embodiments, the training module 316 creates the labeled dataset based on spectral information generated by one or more imaging instruments (e.g., spectrometers). In other embodiments, the training module 316 acquires the labeled dataset—or various spectral information with corresponding labels to be compiled into the labeled dataset—from a datastore. The datastore could be maintained in the memory 304, or the datastore may be accessible via the communication module 308.

As mentioned above, the breadth and depth of the labeled dataset may depend on the intended application of the model. As an example, a user may be interested in creating a model that is able to distinguish between different blend rates of cotton with high accuracy. In such a scenario, the labeled dataset may cover several dozen or hundred cotton blend ratios (e.g., 100 percent cotton, 95 percent cotton and 5 percent polyester, 90 percent cotton and 10 percent polyester, 90 percent cotton and 5 percent polyester and 5 percent nylon, etc.). Such a model may be referred to as a “specialized model” since it is trained to surface insights into a single characteristic. As another example, a user may be interested in creating a model that is able to distinguish between different fibers. In such a scenario, the labeled dataset may cover various fibers (e.g., cotton, polyester, rayon, nylon, etc.) and, in some embodiments, different blend ratios of those various fibers. Such a model may be referred to as a “generalized model” since it is trained to surface insights into multiple characteristics.

At some point, the analysis platform 312 may receive input that is indicative of a request to use one or more trained models. As an example, the analysis platform 312 may receive input indicative of a selection of a textile for which spectral information is available. Alternatively, the analysis platform 312 may receive input that is indicative of a selection of the spectral information itself. In response to receiving the input, the characterizing module 318 can apply a trained model to the spectral information, so as to produce an output. As mentioned above, the trained model may be one of multiple trained models that are applied to the spectral information, in which case multiple outputs may be produced. Multiple outputs could also be produced if the trained model is a generalized model having a multiclass architecture. For example, the trained model could be representative of a neural network that has multiple “branches,” and each of the multiple branches may be designed and then trained to produce an output related to a different characteristic (e.g., fiber, blend ratio, age, wear-and-tear level, etc.).

The GUI module 320 may be generate interfaces through which the output of the trained model or analyses of the output can be posted for review. The interfaces may be viewable using the display mechanism 306 of the computing device 300, or the interface may be viewable on another computing device—in which case the output or analyses of the output may be transmitted to the other computing device using the communication module 308.

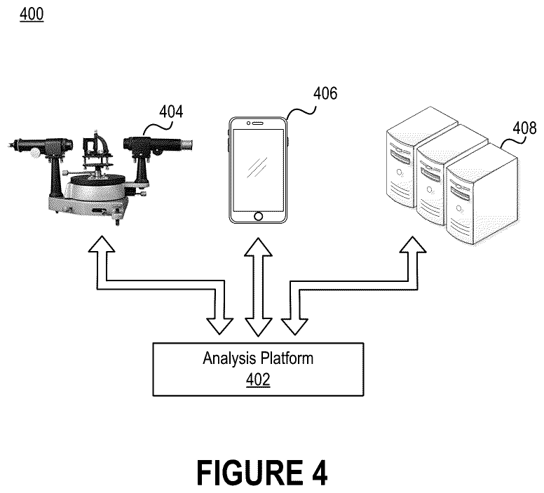

FIG. 4 depicts an example of a communication environment 400 that includes an analysis platform 402 that is configured to obtain data from one or more sources. Here, the analysis platform 402 may obtain data from a spectrometer 404, mobile phone 406, or server system 408 (collectively referred to as the “networked devices”). For example, the analysis platform 402 may obtain spectral information associated with a textile having unknown characteristics from the spectrometer 404. As another example, the analysis platform 402 may obtain a labeled dataset—or various spectral information with corresponding labels to be compiled into the labeled dataset—from the server system 408.

The networked devices can be connected to the analysis platform 402 via one or more networks. These networks can include PANs, LANs, WANs, MANs, cellular networks, the Internet, etc. Additionally or alternatively, the networked devices may communicate with one another over a short-range wireless connectivity technology. For example, if the analysis platform 402 resides on the mobile phone 406, data may be obtained from the spectrometer 404 over a Bluetooth communication channel while data may be obtained from the server system 408 over the Internet via a Wi-Fi communication channel. As another example, if the analysis platform 402 resides on the server system 408, data may be obtained from the spectrometer 404 over the Internet via a Wi-Fi communication channel.

Embodiments of the communication environment 400 may include a subset of the networked devices. For example, the communication environment 400 may include an analysis platform 402 that obtains, in real time, data from the spectrometer 404 as that data is generated via analysis of a textile. Additional data could be obtained from the other networked devices on a periodic basis (e.g., daily or weekly).

Note that the communication environment 400 could also include additional networked devices. For example, the communication environment 400 may include an analysis platform 402 that is able to receive spectral information from multiple imaging instruments. These imaging instruments may include multiple spectrometers, for example.

### Overview of Automated Approached to Determining Fiber Composition

Details regarding an approach to intelligently detecting the fiber composition of textiles are set forth below. For the purpose of illustration, the approach has been described in a specific context, namely, training and then implementing a model to determine fiber composition for recycling purposes. However, those skilled in the art will recognize that aspects of the approach may be similarly applicable to other characteristics and contexts. Thus, the approach may be similarly applicable to training and then implementing a model to determine blend ratio, treatments, colors, ages, wear-and-tear levels, thicknesses, layer counts, and the like.

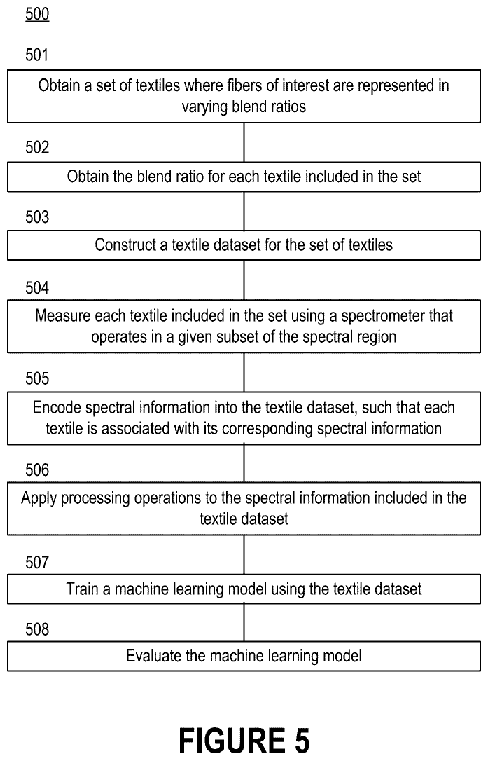

FIG. 5 includes a flow diagram of a process 500 for training and then implementing a model to determine fiber composition. As input, an individual may obtain a set of textiles where fibers of interest are represented in varying blend ratios (step 501). These textiles can be acquired, for example, from retailers, or these textiles can be collected from thrift stores or collection facilities. The source is generally not germane, though there may be benefits to acquiring textiles from more than one source as that may result in the textiles covering a larger gamut of fibers. Normally, the size of the set is based on the number of fibers of interest. For example, if the individual is interested in training a model to distinguish between different cotton blends through spectral analysis, then the individual may obtain one or more textiles at each cotton blend of interest. However, if the individual is interested in training a model to distinguish between several fibers (and combinations of those fibers), then the set may include tens, hundreds, or thousands of textiles.

As mentioned above, the textiles included in the set are selected, acquired, or otherwise obtained so as to cover the fibers of interest. Examples of fibers include cotton, linen, rayon, Tencel lyocell, modal, viscose, polyester, acrylic, elastane, nylon, wool, cashmere, and silk. The textiles included in the set may also include combinations of different fibers in different ratios (e.g., 100 percent cotton, 50 percent cotton and 50 percent polyester, 30 percent rayon and 70 percent nylon). Advantageously, because the approach relies on spectral analysis as further discussed below, the textiles included in the set may be any usage state and any amount of wear. Accordingly, the textiles may be minimally used (i.e., in a “new” state), or the textiles may be thoroughly used (i.e., in a “used” state), for example, by being torn or faded. Similarly, the textiles could be comprised of a single layer of fibers, or the textiles could be comprised of multiple layers of fibers. Thus, the textiles could be “layered.”

A core aspect of the approach is appropriate training a model to distinguish between different fiber compositions. Note that the terms “fiber composition” and “blend ratio” may be used interchangeably to describe the relative proportional amounts of fibers included in a given textile. Those skilled in the art will recognize that, as part of the training process, the user could teach the model to distinguish between different cotton compositions (e.g., 60 percent, 70 percent, 80 percent,.90 percent, and 100 percent) through analysis of spectral information associated with a single texture corresponding to each cotton composition. Alternatively, the user could obtain multiple textiles (e.g., tens or hundreds) at each cotton composition, in order to make the model more malleable following deployment. Models are often deployed in different settings that those in which the models are trained and validated, posing a challenge. To address this challenge, a broad and/or deep swath of textiles may be obtained by the individual.

As part of the training process, an analysis platform that is responsible for training the model may need to obtain the blend ratio for each textile included in the set discussed above (step 502). The blend ratio can be obtained in several different ways. In some embodiments, the blend ratio is determined through visual analysis of a label (also referred to as a “tag”) that is appended to each textile included in the set. Additionally or alternatively, the blend ratio may be determined using robust spectral imaging techniques such as mass spectrometry. In some embodiments, spectral or chemical analysis, for example, by a spectrometer, is performed as a confirmation of the blend ratio as determined through visual analysis. Accordingly, the blend ratio may be determined through spectral or chemical analysis instead of, or in addition to, visual analysis of each textile. Generally, the process of collecting spectral information for the textiles included in the set is partially, if not entirely, automated. For example, while an individual may be responsible for acquiring—or at least identifying—the textiles to be used for training, the analysis platform may generate spectral information for each of the textiles and, as further discussed below, record that spectral information in a data structure along with labels indicating one or more characteristics of each of the textiles.

### Step 1: Create Textile Dataset

Initially, the analysis platform can construct a textile dataset for the set of textiles (step 503). For each textile, the textile dataset may include an entry that specifies (i) the corresponding blend ratio and (ii) corresponding spectral information. As discussed above, the blend ratio of each textile can be determined through visual analysis (e.g., of a label) and/or spectral analysis. In some embodiments, the fiber type ratio is used instead of the blend ratio. In such embodiments, the fiber type ratio may be inferred from the blend ratio. Assume, for example, that a textile (e.g., an article of clothing such as a shirt) is determined to have a blend ratio of 40 percent cotton, 20 percent rayon, 35 percent polyester, and 5 percent elastane. In this situation, the analysis platform may label the textile as having a fiber type ratio of 60 percent cellulosic and 40 percent synthetic since cotton and rayon are cellulosic fibers while polyester and elastane are synthetic fibers. Some embodiments of the analysis platform are programmed or trained to distinguish between several different fiber types, namely, cellulosic (i.e., plant-based) fibers, synthetic (i.e., petroleum-based) fibers, and animal (i.e., protein-based) fibers. There should preferably be a variety of different fibers and blend ratios (or fiber type ratios) that are well represented in the textile dataset to ensure that the model is able to properly generalize.

### Step 2: Collect Spectral Information

Each textile included in the set can then be measured using a spectrometer (e.g., a NIR spectrometer) that operates in some subset of the spectral region between 800 nanometers (nm) and 2,500 nm (step 504). This can be done manually (i.e., with the assistance of an individual) or automatically (e.g., using a robotic computing system that is controlled by the analysis platform). Thus, the analysis platform may be implemented on—or accessible to—a robotic system that is able to cause the textiles included in the set to be measured using a spectrometer, for example, using a pick-and-place mechanism such as a robotic arm. The spectrometer may produce a single spectrum or multiple spectra (e.g., via hyperspectral imaging). Thus, the spectrometer may measure one or more spectrums for each textile included in the set. Normally, the integration time of the spectrometer is set between 0.01 microseconds (ms) and 100 ms, though the integration time may be based on the spectrometer itself and lighting situation.

During analysis of a textile, ambient light is typically blocked from the spectrometer, for example, using a dark, opaque covering. Meanwhile, one or more illuminants may be used to illuminate the textile. Halogen lamps are an attractive choice as the illuminant since the light emitted by halogen lamps will not disrupt spectral measurements in the near-infrared region. Other types of illuminants may be preferred depending on the electromagnetic region of interest.

Preferably, the spectrometer should have a resolution between 2 nm and 15 nm. The resolution could be higher (i.e., less than 2 nm) or lower (i.e., more than 15 nm) depending on the desired precision in accurately detecting fiber composition, available computational resources, etc. To ensure sufficient spectral resolution, the spectrometer may detect across at least 64 channels. While more channels will permit textiles to be spectrally distinguished with greater accuracy, more channels will result in more spectral information, which will in turn require greater computational resources to handle. More than 1,024 channels generally is not necessary.

To improve measurement accuracy, the spectrometer may be calibrated on a periodic basis. While the spectrometer could be recalibrated before every measurement, more commonly the spectrometer is recalibrated before the textiles in a set are analyzed and/or after measurement of a predetermined number of textiles (e.g., 50, 100, 500, 1,000). The spectrometer can be recalibrated in several ways. First, the spectrometer may measure from a white sample (e.g., a white tile) to establish the maximum possible reflectance. Second, the spectrometer may measure when there is no light shone to establish the noise from the spectrometer. The spectra measured in accordance with the first approach may be referred to as the “white calibration spectra,” while the spectra measured in accordance with the second approach may be referred to as the “dark calibration spectra.”

As mentioned above, one or more spectrums may be measured for each textile using the spectrometer. Together, these spectrum(s) are representative of spectral information of the corresponding textile. Spectral information can be encoded into the textile dataset, such that each textile is associated with its corresponding spectral information (step 505). Said another way, the spectral information for each textile may be added as a feature in the textile dataset in the form of spectral data.

Note that, in some embodiments, at least some of the textiles are measured multiple times. Thus, for a given textile, multiple spectra may be produced. These multiple spectra may be averaged together prior to transformation as discussed below with reference to Step 3. Alternatively, predictions produced by the model may be averaged together as part of training or evaluating as discussed below with reference to Steps 4-5.

### Step 3: Transform Raw Spectral Information

Various processing steps can be applied by the analysis platform to the spectral information in its “raw” form (step 506). Because these processing steps are performed before the textile dataset is used for training, these processing steps may also be referred to as “pre-processing steps.”

As an example, the analysis platform may adjust the spectral information using the aforementioned white and dark calibration spectra. Specifically, the analysis platform may adjust each measured spectra in accordance with the following equation:

$${R}_{n} = - ( \frac{R - {R}_{d}}{{R}_{w} - {R}_{d}} ) ,$$

where R is the measured spectra, R_{w} is the white calibration spectra, and R_{d} is the dark calibration spectra.

As another example, the analysis platform may normalize the raw spectral information so that the amplitudes of each spectrum are within a predetermined range of values (e.g., 0 and 1, 0 and 10, 0 and 100). As another example, the analysis platform may reduce noise by smoothing the raw spectral information. For instance, the raw spectral information may be smoothed using a filter (e.g., a Savitzky-Golay filter) that has a passthrough window (or simply “window”) with predetermined bounds (e.g., 5 and 100).

If there are a large number of features in the textile dataset, then it may be reasonable to reduce dimensionality. This can be helpful as a means of conserving computational resources, as well as quickening training. To reduce dimensionality, the analysis platform may employ a method such as PCA.

Depending on the “rawness” of the spectral information—and the desired state of the spectral information for training-various pre-processing operations may be performed by the analysis platform. Examples of pre-processing operations include:
PCA that reduces the dimensionality of the spectral information.
Digital filtering (e.g., with a Savitzky-Golay filter) in which one or more mathematical operations are performed on the spectral information to reduce or enhance aspects of that spectral information.
Derivative analysis of the first derivative or second derivative of the spectral information or analysis of the spectral information.
Standard normal variate computation in which the normal variate is established with mean μ=0 and standard deviation σ=1 with a probability density function of

$$f ( z ) = ( \frac{1}{\sqrt{2 π}} ) {e}^{- z2 / 2} ,$$

where −∞<z<∞.
Detrending in which the effects of a trend are removed from the spectral information to show only the differences in values from the trends, such that patterns can be more readily identified.
Mean centering that is implemented as an additive transformation of a variable. Specifically, as part of a mean centering operation, the analysis platform may substrate the mean of a variable from each value of that variable.
Multiplicative scatter correction that is meant to address scattering of light to account for noise in the spectral information. Multiplicative scatter correction tends to perform better if an offset correction is carried out first. For example, for each wavelength in the spectral information, the analysis platform may compute:

$${x}_{i} = a + {by}_{average} + ε ,$$

where
xᵢ is the i^{th} spectrum of the spectral information used for collection, and
a, b are estimated, for each wavelength by ordinary least squares regression of spectrum xᵢ versus y_{average} over the available wavelengths j.

For each value xᵢⱼ of the corrected spectrum, multiplicative scatter correction can be calculated as follows:

$${x}_{ij} ( M S C ) = \frac{{x}_{ij} - a}{b} ,$$

where
xᵢⱼ is the intensity of the i^{th} spectrum and j^{th} wavelength of the spectral information used for calculation.
Orthogonal signal correction in which data is removed from the spectral information prior to developing the model. Assume, for example, that the spectral information is in the form of a matrix X. As part of an orthogonal signal correction operation, variation may be removed from the matrix X that is orthogonal to a response matrix Y.
Wavelet transformation or compression in which the spectral information is decomposed into a set of wavelets. The analysis platform could employ a continuous wavelet transform in which every possible wavelet over a range of scales and locations are used. A definition of a continuous wavelet transform is set forth below.

$$T ( a , b ) = \frac{1}{\sqrt{a}} {\int }_{- ∞}^{∞} x ( t ) Ψ * \frac{( t - b )}{a} dt$$

Alternatively, the analysis platform could employ a discrete wavelet transform in which a finite series of wavelets (i.e., defined at a particular frequency, or defined at particular locations) are used. A definition of a discrete wavelet transform is set forth below.

$${T}_{m , n} = {\int }_{- ∞}^{∞} x ( t ) {Ψ}_{m , n} ( t ) dt$$

Net analyte signal calculation in which wavelengths are selected via multivariate factor-based hybrid linear analysis, for example,

Outlier removal through visual analysis or statistical analysis may also be helpful in reducing dimensionality. Various approaches have been developed for outlier removal (also called “anomaly removal”).

For example, the analysis platform may implement a local outlier factor (“LOF”) algorithm to the spectral information. In brief, the LOF algorithm is an unsupervised anomaly detection technique that computes the local density deviation of each datapoint with its neighboring datapoints and then classifies as outliers the datapoints that have a substantially lower density than their neighbors.

As another example, the analysis platform may implement a one-class support vector machine (“SVM”) to the spectral information. In brief, the one-class SVM is an unsupervised anomaly detection technique that learns the ability to differentiate datapoints of a particular class from other classes. One-class SVM relies on the general concept of minimizing the hypersphere of the particular class in the training dataset and considering all datapoints outside of the hypersphere to be outliers or out of the training dataset distribution.

As another example, the analysis platform may implement an isolation forest to the spectral information. In brief, the isolation forest is an unsupervised anomaly detection technique that detects anomalies based on isolation, namely, by establishing how far each datapoint is from the rest of the spectral information. The isolation forest may explicitly isolate anomalies using binary trees.

### Step 4: Train Model

The analysis platform can then train a model using the textile dataset (step 507). As mentioned above, the textile dataset may be associated with a set of textiles, and for each textile included in the set, a corresponding entry in the textile dataset may specify (i) the corresponding blend ratio and (ii) corresponding spectral information. The model can learn how to associate spectral information with blend ratios through training on the textile dataset.

The model may be a regression model or classification that is trained on the textile dataset using supervised learning. The regression model or classification model may be based on, for example, a random forest, neural network, dense or deep neural network, linear regression, or support vector machine. Additional processes may be used to facilitate learning in some embodiments. For example, the analysis platform may employ principal component analysis, nearest neighbor analysis, linear discriminant analysis, quadratic discriminant analysis, evolutionary computation, projection pursuit, and the like. In some embodiments, the analysis platform trains ensembles of these different regression models and these processes to identify the optimal combination for determining fiber composition through spectral analysis.

The model can be easily designed so that its output is constrained to a desired range. In fact, many models are designed so that outputs are between 0 and 1. As an example, this can be accomplished using a sigmoid layer in a neural network, This constraint may allow the model to more easily learn fractional representations of percentages, rather than percentages themselves. As such, it may be desirable for the blend ratio for each textile to be represented as fractions rather than percentages. If, for example, a textile is determined to be 60 percent cotton and 40 percent polyester, then the analysis platform may record the blend ratio (e.g., in a data structure that is representative of the textile dataset) as 0.6 cotton and 0.4 polyester. Fraction representations allow different blend ratios to be more readily related and compared across the entire textile dataset.

The model may be designed to accept, as input, feature vectors that specify the (i) blend ratio and (ii) spectral information for different textiles. The term “feature vector” may refer to the output that is generated by the spectrometer in vector form. Other information could also be included in the feature vectors, such as an indication of color, treatment, age, wear-and-tear level, thickness, layer count, etc. For example, indicating the color may be helpful when determining the fiber composition of a black textile versus a white textile of a given blend ratio. Supplementing the blend radio and spectral information with additional information may be helpful in allowing the model to learn, and then distinguish, between different combinations of features (e.g., blend ratio plus wear-and-tear level, layer count and wear-and-tear level, age and thickness, etc.).

Generally, the spectrometer works by radiating light at wavelengths in a spectral range (e.g., 800 to 2500 nm for an NIR spectrometer) at a predefined interval (e.g., 5 nm or 10 nm). The spectral range and predefined interval are specific to the spectrometer, and therefore may vary if different spectrometers are used. The chemical composition of a textile under examination will determine how much of this radiated light is absorbed. The spectrometer is responsible for measuring the amount of radiated light that is reflected back (i.e., not absorbed) by the textile at each wavelength. These measurements may be encoded in a vector-sometimes called a “feature vector” or “spectral vector”—that can be used by the analysis platform. As an example, the vector may specify the amount of radiated light that is reflected at 800 nm, 805 nm, 810 nm, etc.

Through training with the textile dataset, the model can learn how to produce, as output, a predicted fiber composition for a feature vector corresponding to a textile having unknown fiber composition that is provided as input. The predicted fiber composition may be representative of a predicted blend ratio or a predicted fiber type ratio, depending on the nature of the textile dataset. If the model is trained using blend ratios or fiber type ratios in fractions rather than percentages, then the output produced by the model may also be in fractions. Accordingly, the model may be configured to produce, as output, a predicted blend ratio or a predicted fiber type ratio as fractions of the textile for which information is provided as input.

### Step 5: Evaluate and Implement Model

As part of the training process, the analysis platform may evaluate the model (step 508). This can be done in near real time (e.g., as the model is being trained) using a portion of the textile dataset. For example, the analysis platform may use roughly 80 to 90 percent of the textile dataset for training purposes and roughly 20 to 10 percent of the textile dataset for evaluating purposes. Alternatively, the analysis platform may use the entire textile dataset for training purposes and then evaluate the model using an entirely different set of textiles. This other set of textiles may be referred to as the “testing dataset” or “evaluating dataset.”

When evaluating the model, the spectrometer should measure one or more spectrums from each textile as discussed above. Optionally, the textiles can be shredded, sliced, or otherwise fragmented into pieces and then compacted into a “clump” before being measured by the spectrometer to enable more accurate readings of multi-layer clothing that the spectrometer is not able to fully penetrate. Fragmentation following by compaction of the textile may also help ensure that the surface measured by the spectrometer is roughly level. These pieces are generally less than 5 square centimeters (cm2), though the pieces can be larger or smaller depending on the device used for fragmenting, the nature of the textile, the number of layers, etc.

The textile can then pass beneath the spectrometer—in its “whole form” or “fragmented form”-on a transfer mechanism. One example of a transfer mechanism is a conveyor. As the textile passes beneath the spectrometer, the spectrometer can measure the spectrum(s) and then transmit those spectrum(s) to the analysis platform for analysis. The analysis platform may be executed by a computing device that is communicatively connected to the spectrometer, or the analysis platform may be executed by the spectrometer itself.

Thereafter, the analysis platform can provide the spectrum(s) to the trained model as input. As output, the trained model can produce a predicted fiber composition for the textile. As mentioned above, the predicted fiber composition may be representative of a predicted blend ratio or a predicted fiber type ratio. In some scenarios, the textile is one of multiple textiles that are examined by the spectrometer. In some embodiments, the spectrum(s) measured for each textile of the multiple textiles are transmitted to the analysis platform on a continual basis. For example, the spectrum(s) may be transmitted to the analysis platform immediately upon measurement by the spectrometer. In other embodiments, the spectrum(s) are transmitted to the analysis platform on a periodic basis. For example, transmission may be initiated by the spectrometer responsive to a determination that a predetermined number of spectra are available, or transmission may be initiated by the spectrometer at a predetermined cadence (e.g., every 2 minutes, 5 minutes, 10 minutes, or 30 minutes).

As mentioned above, multiple spectra may be measured by the spectrometer for a single textile, and these newly measured spectra may undergo the same pre-processing as the existing spectra used to train the trained model. In such embodiments, the trained model will generate multiple outputs, each of which is representative of a predicted fiber composition that is based on a corresponding one of the multiple spectra. In this situation, the analysis platform may combine the multiple outputs in order to determine an appropriate overall prediction for the textile. For example, the analysis platform may deterministically average the multiple outputs in order to determine the predicted fiber composition. Averaging, aggregating, or otherwise combining the multiple outputs may be most useful when analyzing textiles that cannot be considered homogeneous.

The output produced by the model may be helpful in establishing the nature of a textile with an unknown fiber composition. Those skilled in the art will recognize that the output could be used in various ways, depending on why the textile is being analyzed to begin with. Assume, for example, that the model is applied to textiles with unknown fiber compositions that are to be recycled. In such a scenario, the outputs produced by the model may be used to sort the textiles. More specifically, for each textile to be recycled, the output produced by the model may be used to sort that textile amongst various collections of textiles having different blend ratios, such that each textile is collocated with other textiles having a comparable blend ratio.

Moreover, the analysis platform may indicate how textiles were sorted based on outputs produced by the model. For example, the analysis platform may indicate how each textile was sorted by populating a corresponding entry in a data structure (e.g., with “Bin 001” for 100 percent cotton, “Bin 002” for 90 percent cotton and 10 percent polyester, etc.). Thus, the analysis platform may create, populate, and manage a data structure that not only serves as a digital record of predictions or determinations made by the model, but also indicates how the respective textiles were handled.

The data structure could also be used as input for further processes. Again, assume that textiles whose fiber compositions are being predicted are to be recycled using a recycling system. In this situation, the data structure could be used by the recycling system to determine whether to initiate a recycling operation. For example, the recycling operation may be initiated for textiles having a given fiber composition responsive to determining, based on the data structure, that at least a predetermined number of textiles having the given fiber composition are available for recycling. As another example, the recycling operation may be hastened or slowed based on the number of textiles that are presently available for recycling, as determined from the data structure.

### Methodologies for Training and Implementing a Model

As discussed at length above, the analysis platform may not only train a model to predict characteristics of textiles via spectral analysis, but may also implement the model to predict a characteristic of a textile having unknown characteristics.

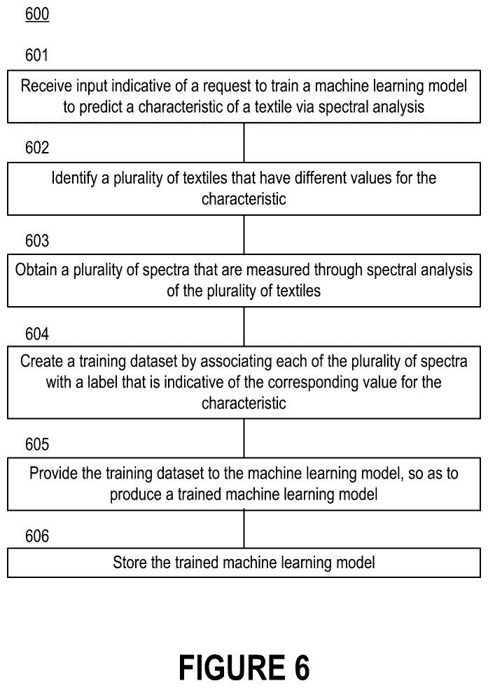

FIG. 6 includes a flow diagram of a process 600 for training a model to predict a characteristic of a textile via spectral analysis. As mentioned above, examples of characteristics include fiber composition, blend ratio, treatment, color, age, wear-and-tear level, thickness, and layer count. In some embodiments, the model is trained to predict combinations of these characteristics. As an example, the model may be trained to predict the wear-and-tear level of textiles that are predicted to have a given blend ratio.

Initially, an analysis platform can receive input indicative of a request to train a model to predict a characteristic of a textile via spectral analysis (step 601). This input may be provided by an individual through an interface generated by the analysis platform. For example, the individual may select a digital element labeled “Train Model” or “Create Model.” Thereafter, the analysis platform can identify a plurality of textiles that have different values for the characteristic (step 602). In some embodiments, the plurality of textiles are identified as a result of the individual providing or obtaining spectral information for each textile. For example, the individual may spectrally image each of the plurality of textiles using a spectroscopy instrument, and the analysis platform may obtain the spectral information generated by the spectroscopy instrument. In other embodiments, the plurality of textiles may have already been spectrally imaged. In such embodiments, the individual may select the plurality of textiles through an interface generated by the analysis platform, and the analysis platform may obtain the corresponding spectral information (e.g., from a database) in response to the selections being made. Thus, the analysis platform can obtain a plurality of spectra that are measured through spectral analysis of the plurality of textiles (step 603).

Then, the analysis platform can create a training dataset by associating each of the plurality of spectra with a label that is indicative of the corresponding value for the characteristic (step 604). For example, the analysis platform may populate the plurality of spectra into a data structure (e.g., a table) and then for each of the plurality of spectra, populate a label that specifies the corresponding value for the characteristic. Assume, for example, that the characteristic is blend ratio. In such a scenario, one or more spectra may be associated with a first blend ratio (e.g., 100 percent cotton), one or more spectra may be associated with a second blend ratio (e.g., 90 percent cotton and 10 percent polyester), one or more spectra may be associated with a third blend ratio (e.g., 80 percent cotton and 20 percent polyester), etc. The analysis platform can then provide the training dataset to the model as input, so as to produce a trained model (step 605). After training (and in some embodiments, testing or validating) is complete, the analysis platform can store the trained model in a storage medium (step 606). Generally, the analysis platform labels the trained model to indicate which characteristic(s) are predictable upon being applied to spectral information of an unknown textile. For example, the analysis platform may title the trained model to specify the characteristic(s), or the analysis platform may append metadata to the trained model that specifies the characteristic(s).

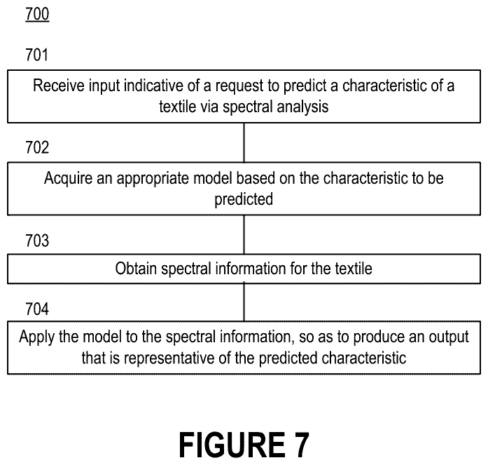

FIG. 7 includes a flow diagram of a process 700 for implementing a trained model to predict a characteristic of a textile via spectral analysis. Initially, an analysis platform can receive input indicative of a request to predict a characteristic of a textile (step 701). For example, an individual may select a digital element labeled “Employ Model” or “Predict Characteristic” that is accessible on an interface generated by the analysis platform. Upon receiving the input, the analysis platform may acquire an appropriate model based on the characteristic to be predicted (step 702). As mentioned above, the analysis platform may train various models to predict different characteristics (and combinations of characteristics), and therefore in order to predict a given characteristic, the analysis platform may acquire the corresponding trained model from a storage medium in which the trained models are stored.

Further, the analysis platform may obtain spectral information for the textile that has unknown characteristics (step 703). For example, the individual may acquire the textile and then spectrally image the textile using a spectroscopy instrument. As another example, the analysis platform may reside on, or be accessible to, a robotic computing system that upon acquiring the textile, is able to spectrally image the textile using a spectroscopy instrument. Regardless of how the spectral information is obtained, the analysis platform can apply the trained to the spectral information, so as to produce an output that is representative of the predicted characteristic (step 704).

As mentioned above, the input may request that multiple characteristics be predicted in some embodiments. In such embodiments, the analysis platform may apply a single model that outputs multiple predictions, either simultaneously (e.g., a single prediction of 100 percent cotton and moderate wear-and-tear level) or sequentially (e.g., a first prediction of 100 percent cotton and a second prediction of moderate wear-and-tear level, or vice versa), or the analysis platform may apply multiple models that each output a single prediction.

### Processing System

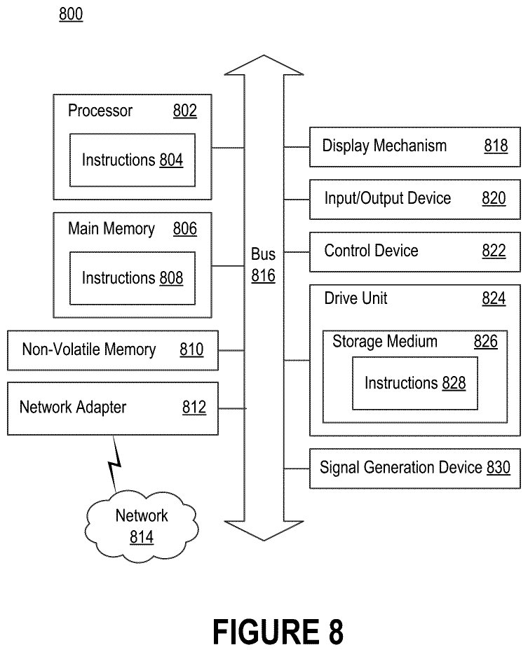

FIG. 8 includes a block diagram illustrating an example of a processing system 800 in which at least some operations described herein can be implemented. For example, components of the processing system 800 may be hosted on a computing device on which an analysis platform is stored and executed.

The processing system 800 may include a processor 802, main memory 806, non-volatile memory 810, network adapter 612, display mechanism 818, input/output device 820, control device 822 (e.g., a keyboard, pointing device, or mechanical input such as a button), drive unit 824 that includes a storage medium 826, or signal generation device 830 that are communicatively connected to a bus 816. The bus 816 is illustrated as an abstraction that represents one or more physical buses and/or point-to-point connections that are connected by appropriate bridges, adapters, or controllers. The bus 816, therefore, can include a system bus, Peripheral Component Interconnect (“PCI”) bus, HyperTransport bus, Industry Standard Architecture (“ISA”) bus, Small Computer System Interface (“SCSI”) bus, Universal Serial Bus (“USB”), Inter-Integrated Circuit (“I2C”) bus, or a bus compliant with Institute of Electrical and Electronics Engineers (“IEEE”) Standard 1394.

While the main memory 806, non-volatile memory 810, and storage medium 826 are shown to be a single medium, the terms “storage medium” and “machine-readable medium” should be taken to include a single medium or multiple media that stores one or more sets of instructions 828. The terms “storage medium” and “machine-readable medium” should also be taken to include any medium that is capable of storing, encoding, or carrying a set of instructions for execution by the processing system 800.

In general, the routines executed to implement the embodiments of the present disclosure may be implemented as part of an operating system or a specific computer programs. Computer programs typically comprise one or more instructions (e.g., instructions 804, 808, 828) set at various times in various memories and storage devices in a computing device. When read and executed by the processor 802, the instructions cause the processing system 800 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine—or computer-readable medium used to actually cause the distribution. Further examples of machine—and computer-readable media include recordable-type media such as volatile memory and non-volatile memory 810, removable disks, hard disk drives, optical disks (e.g., Compact Disk Read-Only Memory (“CD-ROMs”) and Digital Versatile Disks (“DVDs”)), cloud-based storage, and transmission-type media such as digital and analog communication links.

The network adapter 612 enables the processing system 800 to mediate data in a network 814 with an entity that is external to the processing system 800 through any communication protocol supported by the processing system 800 and the external entity. The network adapter 612 can include a network adaptor card, a wireless network interface card, a switch, a protocol converter, a gateway, a bridge, a hub, a receiver, a repeater, or a transceiver that includes a wireless chipset (e.g., enabling communication over Bluetooth or Wi-Fi).

### Remarks

The foregoing description of various embodiments of the technology has been provided for the purposes of illustration and description. It is not intended to be exhaustive or to limit the claimed subject matter to the precise forms disclosed.

Many modifications and variation will be apparent to those skilled in the art. Embodiments were chosen and described in order to best describe the principles of the technology and its practical applications, thereby enabling others skilled in the relevant art to understand the claimed subject matter, the various embodiments, and the various modifications that are suited to the particular uses contemplated.

What is claimed is:

1. A method for training a machine learning model to determine composition of textiles, the method comprising:
for each of a plurality of textiles, obtaining a blend ratio that indicates a ratio of fibers included in that textile,
wherein different fibers of interest are represented in varying blend ratios across the plurality of textiles;
for each of the plurality of textiles, obtaining a spectrum that is measured through analysis of that textile;
creating a textile dataset by associating each of the plurality of textiles with (i) the corresponding blend ratio and (ii) the corresponding spectrum;
providing the textile dataset to a machine learning model as training data, so as to produce a trained machine learning model;
appending metadata that specifies the varying blend ratios of the different fibers to the trained machine learning model; and
storing the trained machine learning model in a storage medium.

2. The method of claim 1, wherein the different fibers include cotton, linen, rayon, Tencel lyocell, modal, viscose, polyester, acrylic, elastane, nylon, wool, cashmere, silk, or any combination thereof.

3. The method of claim 1, wherein, for each of the plurality of textiles, obtaining the blend ratio comprises:
determining the blend ratio through visual analysis of a label that accompanies that textile, and
confirming the blend ratio determined through visual analysis with through chemical analysis of that textile.

4. The method of claim 1, wherein, for each of the plurality of textiles, obtaining the spectrum comprises:
performing spectral analysis by a spectroscopy instrument that produces, as output, the spectrum.

5. The method of claim 1, wherein the spectrum is one of multiple spectra obtained for each of the plurality of textiles.

6. The method of claim 1, further comprising:
for each of the plurality of textiles,
normalizing the spectrum measured for that textile to ensure that each amplitude is between a fixed range of values, and
applying a filter to the normalized spectrum to reduce noise.

7. The method of claim 1, wherein the plurality of textiles are selected so as to cover a predetermined range of blend ratios of the different fibers.

8. The method of claim 1, wherein the trained machine learning model is a regression model trained on the textile dataset using supervised learning.

9. The method of claim 1, wherein the trained machine learning model is a classification model trained on the textile dataset using supervised learning.

10. A method for implementing the trained machine learning model of claim 1, the method comprising:
obtaining a textile having an unknown blend ratio;
measuring a spectrum through spectral analysis by a spectroscopy instrument; and
applying the trained machine learning model to the spectrum, so as to produce an output that is representative of a predicted blend ratio.

11. The method of claim 10,
wherein the spectrum is one of multiple spectra measured through spectral analysis by the spectroscopy instrument, and
wherein upon applying the trained machine learning model to the multiple spectra, the trained machine learning model produces multiple outputs.

12. The method of claim 11, wherein each of the multiple outputs is representative of a blend ratio predicted by the trained machine learning model based on a corresponding one of the multiple spectra.

13. The method of claim 11, further comprising:
determining fiber composition of the textile by deterministically averaging the multiple outputs produced by the trained machine learning model.

14. The method of claim 10, further comprising:
fragmenting the textile into fragments; and
compacting the fragments into a clump;
wherein said fragmenting and said compacting are performed prior to said measuring.

15. The method of claim 10, further comprising:
sorting, based on the output, the textile amongst various collections of textiles having different blend ratios, such that the textile is collocated with other textiles having a comparable blend ratio.

16. The method of claim 15, further comprising:
indicating that the textile was sorted into a particular collection of textiles in a data structure.

17. The method of claim 16,
wherein the data structure is used, as input, by a recycling system to determine whether to initiate a recycling operation, and
wherein the recycling operation is initiated for a given collection of textiles when a predetermined number of textiles having the corresponding blend ratio are available for recycling.

18. A non-transitory medium with instructions stored thereon that, when executed by a processor of a computing device, cause the computing device to perform operations comprising:
receiving input indicative of a request to train a machine learning model to predict a characteristic of a textile via spectral analysis;
identifying a plurality of textiles that have different values for the characteristic;
obtaining a plurality of spectra that are measured through analysis of the plurality of textiles;
creating a training dataset by associating each of the plurality of spectra with a label that is indicative of the corresponding value for the characteristic;
providing the training data to the machine learning model, so as to produce a trained machine learning model; and
storing the trained machine learning model.

19. The non-transitory medium of claim 18, wherein the characteristic is fiber composition, blend ratio, treatment, color, age, wear-and-tear level, thickness, or layer count.
